(19) United States
(12) Patent Application Publication
(10) Pub. No.: US 2004/0161447 A1
(43) Pub. Date: Aug. 19, 2004

(54) Liquid foam producing compositions and dispensing system therefor

(76) Inventor: Leonard Paul, Bloomfield, CT (US)

Correspondence Address: MELVIN I. STOLTZ, ESQ., 51 CHERRY STREET, MILFORD, CT, 06460, US

(21) Appl. No.: 10/777,986

(22) Filed: Feb. 11, 2004

Related U.S. Application Data
(63) Continuation-in-part of application No. 10/090,064, filed on Mar. 1, 2002.
Application No. 10/090,064 is a continuation-in-part of application No. 09/783,060, filed on Feb. 14, 2001, now Pat. No. 6,555,508.
(60) Provisional application No. 60/183,307, filed on Feb. 17, 2000.

(51) Int. Cl. A61M 31/00

(52) U.S. Cl. 424/430; 604/275

(57) ABSTRACT

By providing a therapeutic agent in a mixture of surfactants and water, a unique, improved, liquid based foam producing formulation is realized which is useable for a wide variety of applications. In the preferred embodiment, the improved liquid based foaming producing formulations incorporate one or more therapeutic agents which comprise a silver-based composition. As a result, the formulation is useable for a wide variety of medical applications for preventing, treating, or reducing the spread or transmission of bacteria, virus, infections, and the like.

### TECHNICAL FIELD

This invention relates to formulations constructed for being dispensed as a foam product and, more particularly, to improved liquid formulations capable of being dispensed as a foam product for use in a wide variety of alternate applications, including for washing and/or use for medicinal or medical purposes.

### BACKGROUND ART

Recently, the use of liquid soap and/or liquid dispensable products has become extremely popular, with the ease and convenience provided by such products being appreciated by many individuals. However, in spite of the popularity of liquid dispensable products, no widely useable, multi-purpose, effective liquid dispensable product has been developed which is capable of being dispensed as a foam mousse product to provide consumers with the substantially increased benefits inherent in a foam mousse, while also being useable for medical applications.

By providing improved formulations which are capable of being dispensed from a desired container as a foam mousse, consumers enjoy a wide variety of substantially enhanced benefits. In using any liquid dispensable product, the consumer is required to place a desired amount of the product in one's hands or on the area to be washed, and then vigorously rub the product in the hands or target site in order to develop a lather or foam, in order to achieve the desired cleaning or application. However, by dispensing the product as a foam mousse directly from the container, ease of application and use of the product is enhanced.

A further benefit achieved from dispensing cleaning and medicinal products as a foam mousse is a substantial reduction in the quantity of the product that must be dispensed at any particular time for any desired purpose. The foam mousse is produced by intermixing air into the formulations to produce the foam mousse product being dispensed. As a result, substantially less product is consumed at any particular time, thereby saving the consumer a substantial expense by controlling the amount of product being dispensed and thereby preventing unwanted wasting of product.

Another area in which no effective prior art products have been developed is the medical area wherein numerous applications for a foam delivery system exists with no solution being provided. Furthermore, in most of these areas, medicinal or therapeutic ingredients incorporated into the foam delivery system would greatly enhance their use. However, no prior art product has been made which is capable of meeting this long-felt need. Some specific uses for a product of this nature are diseases and irritations in the vagina and/or the rectum where numerous problems exist that have been largely ignored. Although a long felt need has existed in these areas for safe and effective products, no prior art product has been achieved which is readily available and highly effective in treating or eliminating such diseases and problems.

Therefore, it is a principal object of the present invention to provide a multi-purpose, highly effective, foam delivery system which is capable of being used in a wide variety of alternate product formulations and delivery systems.

A further object of the present invention is to provide a multi-purpose, highly effective, foam delivery system having the characteristic features described above, which is capable of being dispensed from non-aerosol containers and produces a thick, rich, dense, foam mousse.

A further object of the present invention is to provide a multi-purpose, highly effective, foam delivery system having the characteristic features described above, which incorporates an effective amount of a medical or medicinal ingredient for further enhancing the use and application of the present invention in a wide variety of alternate purposes.

Other and more specific objects will in part be obvious and will in part appear hereinafter.

### SUMMARY OF THE INVENTION

By employing the present invention, all of the difficulties and drawbacks of the prior art have been overcome, and a highly effective, multi-purpose, universally applicable improved liquid based foam delivery system is achieved. In addition to attaining a universally useable, liquid based foam product, the present invention achieves an improved liquid based foam system that can be employed for all normal washing, as well as for treating a wide variety of medical conditions and problems.

The principal feature of the present invention is the attainment of an improved liquid based formulation which is capable of being employed with and dispensed from non-aerosol, foam-producing containers in a consistent and repeatable manner. Typically, conventional liquid formulations are incapable of repeatedly passing through the foam producing dispensing heads associated with foam containers/dispensers. Due to the inherent nature of conventional liquid products, the fine mesh screens employed with foam heads are quickly clogged, preventing the effective, reliable use of conventional liquid products in this manner.

With the present invention, a unique improved liquid based, foam producing formulation is realized which eliminates the prior art inabilities and provides a formulation which is effectively dispensed from foam producing dispensing heads in a consistent, reliable and repeatable manner, free from clogging failures. In accordance with the present invention, the principal ingredients are a mixture of surfactants, which inherently possess foam enhancing or foam producing qualities, and water with the quantity of water employed representing a critical factor. In order to achieve a formulation which is capable of being dispensed as a foam mousse, the total water employed must range between about 40% and 95% by weight of the total weight of the entire composition.

An additional ingredient which is preferably incorporated into the liquid foaming formulations of the present invention comprises one or more therapeutic agents. As is more fully detailed below, by incorporating a therapeutic agent, the present invention achieves a unique, universally employable, foam mousse product which is capable of being used in a wide variety of alternate applications for a wide variety of alternate purposes. In this regard, the presence of a therapeutic agent in the liquid foam formulation substantially enhances the usability and applicability of the liquid foam producing product, while also providing substantially enhanced beneficial results in areas where beneficial results have not been attainable.

In accordance with the present invention, the final required ingredient is one or more surfactants. In general the quantity of the surfactants employed in the composition preferably ranges between about 0.1% and 70% by weight based upon the total weight of the entire composition. It has been found that the combination of the surfactant and water assures the production of the foam mousse in a dependable, repeatable and consistent manner.

The invention accordingly comprises the several steps and the relation of one or more such steps with respect to each of the others, and the article produced possessing the features, properties, and relation of elements which are exemplified in the following detailed disclosure, with the scope of the invention and being indicated in the claims.

### THE DRAWINGS

For a fuller understanding of the nature and objects of the invention, reference should be had to the following detailed description taken in connection with the accompanying drawings, in which:

FIG. 1 is a perspective view of one embodiment of a delivery system incorporating a unique nozzle/cannula of the present invention;

FIG. 2 is a front perspective view of the nozzle/cannula shown in FIG. 1;

FIG. 3 is a bottom perspective view of the nozzle/cannula shown in FIG. 1;

FIG. 4 is a perspective view of a second embodiment of a delivery system incorporating an alternately constructed nozzle/cannula;

FIG. 5 is a cross-sectional side elevation view, partially broken away, depicting a check valve mounted in a bottle;

FIG. 6 is a top plan view of a disk member forming a component of the check valve of FIG. 5; and

FIG. 7 is a cross-sectional side elevation view, partially broken away, of a further alternate embodiment of a one-way delivery system made in accordance with the present invention.

### DETAILED DISCLOSURE

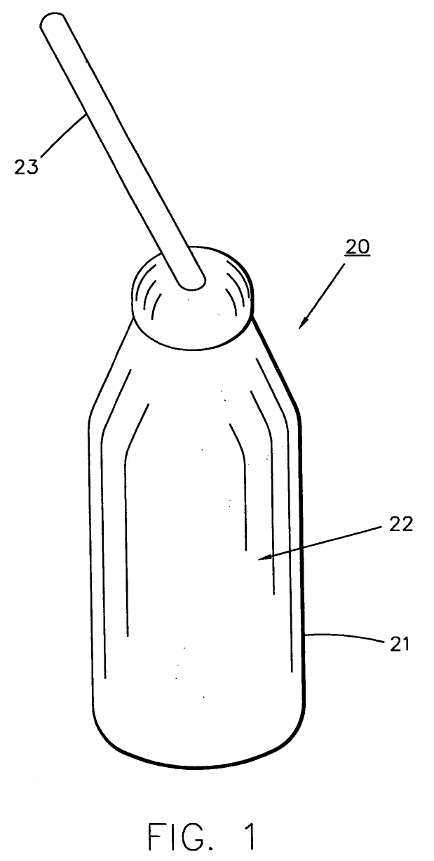

By referring to the following detailed discussion, various preferred compositions and formulations are provided, along with alternate, preferred constructions for delivery containers employable with the formulations of the present invention. In this regard, FIGS. 1-7 depict the preferred embodiments for delivery containers within which the formulations of the present invention can be housed.

It is to be understood, however, that this detailed disclosure is provided for exemplary purposes only, and is not intended as a limitation of the present invention, since further alternate formulations and product constructions can be made without departing from the scope of this invention. Consequently, all of these further alternate embodiments and alternate formulations are intended to be within the scope of the present invention.

As detailed herein, the present invention attains a liquid based, foam producing composition which is capable of being employed in a wide variety of medical applications, as well as in normal, everyday applications wherein increased cleanliness and/or bacterial or antimicrobial cleaning is desired. In addition, by employing the product dispensing containers detailed herein and forming a part of the present invention, a highly versatile, multi-purpose, antimicrobial/antibacterial foam producing dispensing system is realized for virtually eliminating unwanted bacteria and/or microbes.

In accordance with the present invention, the liquid based, foam producing formulations principally comprise a mixture of one or more surfactants, a therapeutic agent, and water. In this regard, the surfactants employed possess foam enhancing or foam producing qualities which combine with the water to achieve formulations capable of being dispensed as a rich, foam mousse. In addition, in order to achieve the desired antimicrobial and/or antibacterial qualities, one or more therapeutic agents are also incorporated into the formulations in sufficient quantities to assure the desired result.

It has been found that by controlling the quantity of the surfactants and the quantity of the water, a consistent, dependable, repeatable foam mousse is produced and dispensed from the desired container. In addition, by incorporating an effective quantity of the desired therapeutic agent or agents, the precisely desired medicinal and/or multi-functional purposes being sought by the formulations are realized in a formulation which is easily dispensed and used by any individual.

In Table I, an overall composition of the present invention is fully detailed. This composition represents the preferred formulation for achieving the goals of the present invention.

TABLE I
Liquid Based Foam Producing Composition

| Ingredient | % by Weight |
|---|---|
| Mixture of Surfactants | 0.1-70 |
| Therapeutic Agent | Effective Amount |
| Water | 40-99.8 |
| pH adjusting agent | As needed |
| Additives | As needed |

In carrying out the teaching of the present invention, it has been found that one or more surfactants are preferably employed, with the surfactants being selected from the group consisting of polysorbate 20, cocoamide DEA, polysorbate 60, polysorbate 80, ammonium or alkaline salts of sulfated aliphatic alcohols, ammonium or alkaline salts of sulfated aliphatic ethoxylated alcohols, cocoamido derivatives, ethoxylated aliphatic phenolics, sarcosinates, sodium lauryl sulfoacetate, sodium lauroyl sarcosinate, and vegetable oil based soaps.

By employing formulations made in accordance with the foregoing teaching, it has been found that a highly effective, multi-purpose, liquid based, foam producing product is achieved. One of the principal features of this formulation is that the pH resulting from this composition is relatively mild for most uses. However, if desired, the pH is easily adjusted to range between about 5.0 and 7.6. As a result, virtual neutrality is attainable and the liquid foam producing product is comfortable for virtually any use or application.

In the preferred embodiment, the improved, multi-purpose liquid foaming formulations of the present invention comprises an effective amount of a therapeutic agent. Typically, the therapeutic agent comprises one or more selected from the group consisting of antiseptic agents, anti-bacterial agents, anti-microbial agents, anti-viral agents, medicines, anti-inflammatory agents, anesthetics, analgesics, and anti-itch agents. Depending upon the particular use desired, one or more therapeutic agents are added to the composition in order to provide the desired enhanced result.

Although the therapeutic agent employed in the liquid, foam producing compositions of the present invention may be selected from a broad category of therapeutic compounds which provide the desired functions detailed above, the following agents comprise a representative sample of the type of agents that has been found to be highly effective in achieving the goals of the present invention. This sample of therapeutic agents comprises one or more selected from the group consisting of triclosan, spirulina, calcium spiruline, nonoxynol-9, benzocaine, lidocaine, silver nitrate solutions, lidocaine-hydrochloride, iodine, povodone-iodine, hot water solutions of spirulan, silver nanocrystals, colloidal silver, and colloidal silver solutions. As detailed below, each of these therapeutic agents provides a particular target area or desirable function for enabling the improved liquid based foam producing compositions of the present invention to be used to attain results previously thought to be unattainable. As will be understood from the disclosure provided herein, each of these therapeutic agents are unique in providing particular targeted results. Consequently, the listing of one or more known agents cannot be employed as a teaching of any other agent contained in the foregoing list.

One product area which typifies a composition of the present invention is the creation of an antiseptic, anti-bacterial, or anti-microbial liquid based, foam producing composition for general, everyday use, and/or for application wherein an antiseptic or anti-bacterial foam is desired. In order to attain a product of this nature, it has been found that by incorporating triclosan as the therapeutic agent, a highly effective, anti-bacterial, antiseptic and/or anti-microbial liquid based foam producing product is realized. In addition, by adding nonoxynol-9 as the therapeutic agent, an anti-viral formation is attained to be used for medicinal or medical purposes in hospitals, nursing homes, and elderly housing for use when water is not available, or for general cleanliness. Furthermore, it has also been found that the use of one or more selected from the group consisting of aqueous solution of silver nitrate, silver nanocrystals, colloidal silver, colloidal silver solutions, and equivalents thereof, as the therapeutic agent creates a formulation which can be employed for treating burn victims as well as providing a multi-purpose and multi-functional anti-microbial, anti-bacterial, anti-fungal, and anti- viral composition, as described and referenced herein.

In Table II, a preferred formulation for using triclosan in the liquid foam producing product of the present invention is detailed, while Table III provides a more detailed formulation, with the specific ingredients for all functions being provided. In formulating this product, the established effective amount of triclosan ranges between about 0.2% and 2.0% by weight based upon the total weight of the composition.

TABLE II
Anti-Bacterial/Antiseptic Foam Producing Product

| Ingredient | % by Weight |
|---|---|
| Mixtures of Surfactants | 5-70 |
| Triclosan | 0.2-2.0 |
| Water | 40-95 |
| pH Adjusting Agent | As needed |

TABLE III
Anti-Bacterial/Antiseptic Foam Producing Product

| Ingredient | % by Weight |
|---|---|
| Polysorbate 20 | 5-30 |
| Cocoamide DEA | 3-10 |
| Ammonium Lauryl Sulfate | 25-40 |
| Triclosan | 0.2-2.0 |
| Water | 40-80 |
| pH Adjusting Agent | (q.s. for pH of 6.5-8.0) |

In achieving an effective, useable and desirable anti-bacterial/antiseptic liquid, foam producing product which employs triclosan, it has been found that polysorbate 20 is preferably employed as a surfactant in order to allow the triclosan to be dissolved in the aqueous solution. Since triclosan is not water soluble, an agent is required to dissolve the triclosan into the solution. Although polysorbate 20 is preferred for this purpose, another equally effective agent may also be used.

In addition, it has also been discovered that the quantity of cocoamide DEA employed in the composition preferably ranges between about 3% and 30% of the quantity employed for the ammonium lauryl sulfate. By employing these parameters, a highly effective, multi-purpose, antiseptic/anti-bacterial liquid based foam producing product is achieved.

In addition to achieving an effective antiseptic/anti-bacterial liquid based foam producing composition, the foregoing composition also possesses a pH of about 6.5 to 8.0. This result is attained by employing the ingredients such as citric acid or equivalent, in the quantities detailed above, along with a small quantity of one or more pH adjusting agents which are well known to those skilled in this art.

Other areas which greatly benefit from the attainment of a liquid based, foam producing composition which incorporates a therapeutic agent are found in a wide variety of medical or medicinal applications. In this regard, various diseases which are caused by viruses have been virtually ignored by prior art products due to the inability of these prior art products to deliver an effective anti-microbial or anti-viral composition directly to the problem site.

The two areas where problems had continuously plagued the medical field and have gone unsolved are found with the diseases and/or irritations which affect the vagina and/or the rectum such as chlamydia or gonorrhea. However, by employing the present invention, these problem areas are quickly and easily resolved.

It has been found that by incorporating an effective amount of an anti-bacterial, anti-microbial, anti-viral, anti-itch, antiseptic, anti-inflammatory, anesthetic, and or analgesic therapeutic agent in the liquid based, foam producing composition of the present invention, a safe and highly effective treatment system is realized for treating various anatomical problems, particularly vaginal and rectal diseases and irritations. In this regard, therapeutic agents such as triclosan, nonoxynol-9, octoxynol-9, silver nanocrystals, colloidal silver, colloidal silver solutions and other equivalent anti-viral or anti-bacterial compositions, can be employed in the liquid, foam producing formulations of the present invention to achieve a resulting product capable of resolving problems that have heretofore been unresolved.

By employing the compositions detailed above for attaining an anti-bacterial, anti-viral, and/or anti-microbial liquid, foam producing product, a safe, and effective delivery system is realized for enabling any individual or healthcare provider to quickly, easily, and conveniently apply the foam product directly to areas which are otherwise incapable of being easily accessed, with complete assurance that both cleaning and anti-viral medication is simultaneously delivered precisely to the site where needed. As a result, by employing the present invention, areas of the body are capable of been effectively treated where presently no effective treatment is available.

It is the intent of this invention to take advantage of the well-known phenomena caused by surface active agents on cohesion and surface tension to make use of the foam mousse product as a medical device when used with a formulation composed of an anti-bacterial or anti-viral drug and in some cases a combination of the two. The anti-bacterial foam compositions that includes Triclosan, silver nanocrystals, colloidal silver, colloidal silver solutions or equivalent silver compositions in the formulation has been produced and has been found to help eliminate bladder infection in women caused by improper or negligent habits when using the toilet.

The pH of the anti-bacterial foam producing cleansing formula had to be lowered to 7 in order to accommodate any tender skin areas. In use, a small amount of the foam mousse interspersed with triclosan, colloidal silver, silver nanocrystals, colloidal silver solutions, or other anti-bacterial, or an anti-viral agent, such as nonoxynol-9, is applied to a toilet tissue before use in cleaning the rectum and/or vaginal area. In this way, the chance of developing this type of bladder infection is substantially reduced. The foam mousse dries rapidly and the film is gentle and does not cause any irritation. If necessary, the film can be removed by simply wetting a toilet tissue and wiping the area thirty seconds after the application of the foam. In the preferred construction, the foam mousse is packagable in a pocketbook size three ounce foamer for added convenience.

There are many other areas in which the anti-bacterial/anti-viral foam producing compositions of this invention can be used as a medical device such as within a hospital setting, the workplace, in the home, the armed services, or any other event in which an open wound of any size is a possibility. In a hospital or surgical setting, protection from viral or bacterial infections is a priority. With the use of a foamer and the proper antiseptic foam, a protective skin is produced that protects the cells surrounding the wound from infection. It follows that the same protective event will follow in any location where an open wound or cut has happened.

During the last several years, substantial attention has been given to the beneficial medicinal qualities provided by silver. In general, it has been found that silver possesses powerful anti-microbial properties, functioning as a natural antibiotic and preventative against infections. It is believed that silver acts as a catalyst to disable enzymes that one celled bacteria, viruses, and fungus need for their metabolism. As a result, the presence of silver near a virus, fungus, bacterium, or any other single cell pathogen effectively disables the oxygen metabolism of the enzyme causing the pathogen to suffocated and die.

In order for silver to achieve a bacteriacidal effect, silver ions must be available, either in solution or dry, at the bacterial surface. If present in the proper form, the silver ions appeared to kill the microorganisms instantly by blocking the respiratory enzyme system.

It has been found that silver can be presented in various different forms in order to achieve the desired anti-microbial effect. In this regard, colloidal silver, nanocrystalline silver, silver nitrate solutions, colloidal sliver solutions, and equivalents thereof have been effectively employed in performing as an anti-microbial agent for fighting and/or eradicating/killing bacteria, viruses, and funguses.

Colloidal silver solutions are a suspension of metallic silver particles (colloidal silver) in purified water. It is colorless, tasteless, odorless, and non-toxic. The product is used by individuals both topically and internally for a wide variety of ailments. Nano-crystalline silver or silver nanocrystals consists of silver which has been processed using nanotechnology to achieve extremely small, nano-sized silver crystals. Typically, nanocrystalline silver is produced in two different forms. One form comprises a nanocrystalline silver film which is coated on a support member, such as a high-density polyethylene mesh. In its other typical form, the silver nanocrystals are exposed to water in which the silver is rapidly released as ions, radicals, and clusters.

One of the principal difficulties in effectively employing silver for its beneficial effects is the difficulty in getting silver into the body where the silver is able to react. Typically, silver is ingested orally, being transported through the mouth into the bloodstream. Although it is believed that the silver will reach and eradicate unwanted bacteria, viruses and funguses, the lack of control over the delivery of silver to the desired pathogen often produces inconsistent results.

In spite of the difficulties that have been experienced in delivering silver to precise locations where its beneficial effects is desired and the common knowledge that silver is an anti-microbial agent for fighting and/or eradicating bacteria, viruses, and funguses, silver has never been employed in any delivery system which produces a foam composition for effectively providing the benefits of silver directly to a precise site or location on the human body or in a cavity of the human body where silver treatment is desired. However, by employing the present invention, this previously inconceivable and unattainable delivery and/or use is now realized, with virtually any desired location being reachable, including both the surface and the interior cavity of the rectum and vagina.

In accordance with the present invention, one or more silver compositions are incorporated into the foam producing delivery systems of the present invention with the silver composition functioning as the therapeutic agent incorporated into the foam producing formulation of the present invention. In this way, the foam producing delivery system of the present invention is capable of providing a system in which silver is delivered directly to the precise location or site where the anti-microbial benefits of silver can be most effectively employed. In addition, the present invention enables users to achieve the application of silver in easily dispensed and easily employed delivery system, both of which have previously been incapable of being attained.

By employing the liquid based, foam producing delivery system of the present invention, the beneficial effects provided by silver compositions is achieved in a unique manner which enables the silver compositions to be placed directly at the precise site where bacteria, funguses, viruses, and the like are present. In this regard, it has been found that the overall liquid based, foam producing composition defined in Table I may be employed with the desired silver composition forming the therapeutic agent. More particularly, it has also been discovered that a highly effective, silver based, multi-purpose, foam producing composition is achieved by employing of the formulation defined in Table IV, wherein the overall formulation for any desired silver based ingredient is provided.

TABLE IV
Silver Based, Multi-Purpose Foam Producing Composition

| Ingredient | % by Weight |
|---|---|
| Silver Based Therapeutic Agent | .00001-2 |
| Surfactant | 0.1-60 |
| Additives | 0-50 |
| Water | q.s. to 100% |

By referring to Table V, one preferred composition made in accordance with Table IV is provided. In this composition, the silver based therapeutic agent comprises colloidal silver.

TABLE V
Colloidal Silver Based, Multi-Purpose Foam Producing Composition

| Ingredient | % by Weight |
|---|---|
| Colloidal Silver Solution | 40-99.8 |
| (10 ppm-32 ppm in water) | |
| Surfactants | 0.1-30 |
| Additives | 0.5-2 |

In this composition, colloidal silver in water is employed, with the silver content ranging between about 10 parts per million and 32 parts per million in a water solution. In addition, the preferred surfactants comprise one or more selected from the group consisting of polysorbate 20, cocoamide DEA, polysorbate 60, polysorbate 80, ammonium or alkaline salts of sulfated aliphatic alcohols, ammonium or alkaline salts of sulfated aliphatic ethoxylated alcohols, cocoamido derivatives, ethoxylated aliphatic phenolics, sarcosinates, sodium lauryl sulfoacetate, sodium lauryl sarcosinate, and vegetable oil based soaps. Furthermore, the additives preferably comprise one or more selected from the group consisting of pH adjusting agents, perfumes, preservatives, and lanolin.

Another particular, specific formulation made in accordance with Table IV is detailed in Table VI. In this formulation, another highly effective, multi-purpose foam producing composition is realized.

TABLE VI
Multi-Purpose Foam Producing Composition

| Ingredient | % by Weight |
|---|---|
| Colloidal Silver Solution | 75 |
| (32 ppm in water) | |
| Vegetable Oil Based Soap | 24 |
| Lanolin | 1 |

A further particular, silver based, multi-purpose, foam producing composition which has been found to be particularly effective in providing a broad-based, highly usable antimicrobial agent for fighting and/or eradicating bacteria, viruses, and funguses precisely at a desired site or location on or in an individual is a detailed in Table VII. In this composition, which comprises a colloidal silver and water solution in combination with sodium lauryl sulfoacetate or sodium lauroyl sarcosinate, a highly effective foam is generated from any desired container, with the silver bearing foam composition being quickly and easily directly applied to a precisely desired site or location where needed, such as locations where infections, viruses, funguses, or other undesirable and unwanted bacteria or diseased causing agents are located.

TABLE VII
Silver Based Foam Producing Composition

| Ingredient | % by Weight |
|---|---|
| Colloidal Silver Solution | 99-99.8 |
| (10 ppm-32 ppm in water) | |
| Sodium Lauryl Sulfoacetate or | .2-1.0 |
| Sodium Lauroyl Sarcosiinate | |

In achieving this multi-purpose, highly effective foam producing composition, any desired sodium lauryl sufoacetate or sodiumlauroyl sarcosinate may be employed. However, it has been found that Lathanol LAL flakes, manufactured by Stepan Company of Northfield Illinois or Hampasil, manufactured by Dow Chemicals of Delaware, are most desirable and produce highly effective compositions when combined with the colloidal silver and water solution. In addition, it has also been found that this formulation achieves a composition which is nontoxic and can be applied directly to open wounds, as well as to delicate, internal sites in the human body, without causing any damage to an individual's tissue cells or membranes.

By employing the silver based, multi-purpose, anti-microbial foam producing composition defined in Table VII, numerous ailments, infections, viruses, and diseases can be effectively controlled and/or eradicated. In particular, it has been found that the use of this composition is effective in treating and/or controlling a wide variety of diseases. One such disease that has been found to be extremely difficult to treat or control is SARS. However, it is believed that by employing the present invention either defensively or offensively the foam composition of this invention is capable of limiting and/or controlling the spread of the SARS virus.

Furthermore, other less catastrophic but troublesome diseases have also been effectively treated using the foam composition of the present invention, and in particular, the formulation defined in Table VII. One such other malady is athlete's foot, which has been found to be virtually eradicated by employing the present invention. In addition, easily transmitted viruses have been found to be controlled and/or eradicated by employing the present invention as a foam for the mouth or as a mouthwash.

In this regard, it has been found that the present invention provides a highly effective direct mouth treatment or mouthwash for killing and/or controlling numerous viruses, bacteria, and the like by merely spraying the foam composition of the present invention into the mouth and then retaining the composition in the mouth for between about one and three minutes. Thereafter, the composition is merely discharged from the mouth by spitting, with the use of any liquids being avoided for about 10 minutes. In this way, effective control and/or elimination of numerous unwanted diseases has been realized.

It has also been found that numerous alternate products can be treated using the foam composition of the present invention to attain a multi-purpose anti-microbial application system. In this regard, the application of the foam composition of the present invention to woven and/or non-woven sheets has been found to be highly effective in attaining anti-microbial wipes which can be employed on an individual's face, hands, or other surfaces, as well as on the skin surface of babies and children, wherever highly effective cleansing and/or antimicrobial treatment is desired. In this regard, pre-applied or preformed sheets of any desired size can be created and dispensed in any suitable manner for enabling the anti-microbial compositions of the present invention to be used in an effective manner.

It has also been found that the silver containing foam composition of the present invention, particularly as detailed in Table IV, can also be applied to face masks used by individuals in numerous diverse settings, such as hospitals, clinics, operating rooms, field operations etc. By spraying the foam composition of the present invention onto conventional face masks, a highly effective, anti-microbial protection zone is established between the breathing passageway of an individual and in the field or environment in which a high-level of toxic and/or infectious bacteria, viruses, diseases, etc. may be present. Furthermore, it has also been found that spraying the foam composition of the present invention on filters used in air circulation systems provides enhanced protection against the inhalation of unwanted airborne toxic and/or infectious bacteria, viruses, diseases, etc. In this way, homes, businesses, airplanes, and other similar areas in which air filtration and/or air conditioning or heating systems are employed can benefit from the continuous removal of unwanted and potentially dangerous airborne particles or microbes.

In addition, the foam composition of the present invention can be sprayed on a carrier sheets, such as woven or non-woven material with the foam bearing material being used in a filter assembly. In this way, the desired anti-microbial protection is attained.

It has also been discovered that colloidal silver solutions can be applied directly onto any desired carrier sheet or support sheet which is then dried to eliminate the water. Although the colloidal silver solution may be applied to these products in various ways, it has been found that colloidal silver solutions are most easily applied by either spraying the colloidal silver solution onto the product or dipping the product into a colloidal silver solution.

Once dried, the colloidal silver particles are retained in the carrier sheet or support sheet, ready for providing the desired antimicrobial effect. By employing the colloidal silver bearing carrier sheet or support sheet in a face mask, filter, or other similar use or application, the desired antimicrobial protection is attained in a wide variety of important areas.

Furthermore, colloidal silver solutions, which are subsequently dried, and/or silver-bearing foam compositions as detailed herein, can also be applied in the manner detailed above to medical devices, such as stints, sutures, bandages, and the like. In this way, added antimicrobial benefits are provided, a the precise sites where such treatment is needed.

In addition to the use of the colloidal silver and colloidal silver based, foam producing composition of the present invention on face masks, air filters, and/or carrier sheets as a preventive treatment against inhalation of unwanted diseases, bacteria, viruses, etc., it has also been found that silver nanocrystals are also effectively used in creating protective face masks, air filters, and carrier sheets. In this regard, the silver nanocrystals are deposited on the material or the filter carrier sheet which is integrally formed as a component of an otherwise conventional face mask and/or air filter.

In this form, the nanocrystalline silver releases ions into environments in order to create the anti-microbial activity. As a result, during the breathing of an individual through the face mask or the movement of air in homes, businesses, airplanes, and the like, the silver ions are released and the anti-microbial activity is realized. In this way, any unwanted bacteria, viruses, funguses, and the like which might otherwise be inhaled by an individual is exposed to the anti-microbial silver ions, and effectively killed and/or controlled.

In addition to employing silver nanocrystals as an integral component of face masks, air filters, and/or carrier sheets, in order to substantially enhance the control achieved over inhaled bacteria, viruses, microbes, etc., silver nanocrystals can also be employed in attaining foam producing compositions. In this regard, by referring to Table VIII, a further composition encompassed by Table IV is provided, wherein an overall, preferred, composition incorporating silver nanocrystals in achieving a foam producing composition is detailed. By employing this composition, the beneficial results detailed above are also realized.

TABLE VIII
Nanocrystalline Based Foam Producing Composition

| Ingredient | % by Weight |
|---|---|
| Silver Nanocrystalline Powder | 0.02-2 |
| Surfactants | 0.05-8 |
| Additives | 10-50 |
| Water | q.s. to 100% |

In addition, by referring to Table IX, a more detailed preferred formulation for a nanocrystalline based foamed producing composition is provided. In this formulation, the specific additives are detailed, along with the preferred quantity range for each additive.

TABLE IX
Nanocrystalline Based Foam Producing Composition

| Ingredient | % by Weight |
|---|---|
| Silver Nanocrystalline Powder | 0.05-1 |
| Propylene Glycol | 4-6 |
| Denatured Ethanol | 15-40 |
| Surfactants | 0.1-5 |
| Water | q.s. to 100% |

In addition to the use of the present invention as detailed above, the foam producing compositions of this invention are also useable as a prophylactic when employed by either the female and the male to enhance protection against the spread of diseases. The female sprays a nonoxynol-9 bearing foam composition, or other proven anti-AID's drug, deep into the vagina or rectum in the case of males. The second barrier would be set up by the male wetting the penis with water and then using the same foam spread around the penis. The formulation is slippery enough to act as a lubricant to enhance the ease of the sexual act.

In a further area, the foam mousse is well suited for burn patients, since it lays down a layer of medicant easily and without irritation. Povidone iodine complex (10% active), a silver nitrate solution (0.5% active), colloidal silver, and silver nanocrystals (0.1% solution) are excellent antiseptics for burn patients. Povidone iodine, silver nitrate, colloidal silver and silver nanocrystals are water based solutions to be sprayed or placed over the affected area as a foam mousse without any rubbing. By employing the present invention, the active therapeutic agent is slowly released onto the skin, providing or assisting in the healing process. As with all burn patients, bandages should not be placed over the affected area.

In order to attain the desired foam mousse from the liquid foam producing composition of the present invention, non-aerosol, unpressurized, foam delivery dispensers known in the art are employed. These dispensers typically comprise a movable, finger-operated dispensing head or cap mounted to a container in which the improved liquid foam producing composition of the present invention is retained. The movable, finger operated dispensing head/cap is constructed to draw the liquid foam producing composition from the container into the cap and force the composition through various screens while intermixing air therewith to produce a dispensed product which comprises a foam mousse.

In an alternate configuration, the foam delivery dispenser comprises a soft pliable bottle in combination with a dispensing cap/head structure which allows the user to squeeze the soft pliable bottle to force the composition in the container to pass through the cap and deliver the desired foam mousse product. Regardless of which structure is employed, the resulting foam mousse product is substantially equivalent and can be employed with equal efficacy in the present invention.

In most applications employing the present invention, the liquid, foam producing composition defined herein is retained in a container incorporating the movable, finger-operated dispensing head/cap. In this way, the desired foam mousse product is quickly and easily dispensed into one's hand for use and application or to any other desired site or location.

Alternatively, in those applications where delivery directly into cavities, such as the vagina, the rectum, or deep wounds are desired, the squeeze bottle construction is preferred. In addition, a soft, pliable, elongated tube or nozzle is mounted to the cap, to enable the foam mousse product to be delivered directly in the particular cavity at the precise location of the inflammation or virus being treated. In this way, direct application is realized with ease and efficiency.

By referring to FIGS. 1-4, along with the following detailed discussion, two alternate embodiments for a delivery system for the present invention are fully detailed. In particular, FIGS. 1-4 and the following detailed discussion focused upon two preferred embodiments for delivering the compositions of the present invention directly into cavities, such as the vagina, rectum, or deep wounds as discussed above.

In FIGS. 1, 4, and 7, three alternate delivery system constructions are depicted, both of which can be employed with equal efficacy. In the embodiment depicted in FIG. 1, delivery system 20 comprises squeeze bottle 21, which contains the desired formulation 22, made in accordance with the present invention. In addition, delivery nozzle or cannula 23 is mounted to bottle 21 in order to deliver the desired formulation directly to the precise location where formulation 22 is desired for optimum efficacy.

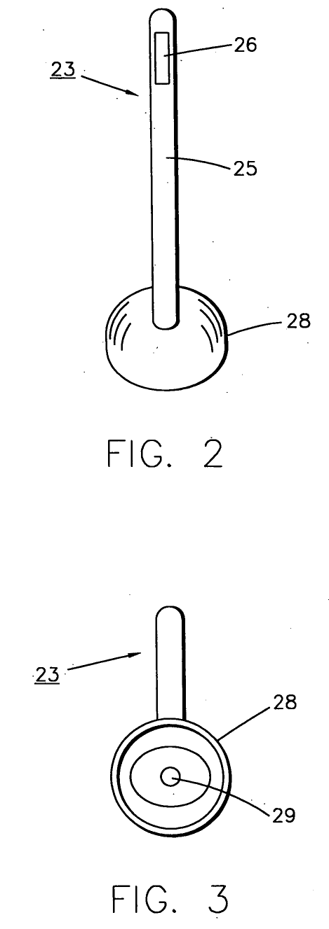

In this regard, delivery nozzle/cannula 23, which is more fully depicted in FIGS. 2 and 3, comprises a uniquely constructed, elongated tube portion 25 which is specifically configured for insertion into desired human cavities, such as the vagina, the rectum, and deep wounds. In this regard, tube portion 25 comprises a precise overall length which assures complete insertion of tube portion 25 in the desired orifice or opening, without injuring the surrounding soft tissue. In addition, tube portion 25 is constructed with maximum flexibility in order to provide ease of use.

Generally, this length preferably comprises between about 2 inches and 3.5 inches. In addition, the distal end of tube portion 25 comprises apertures 26 formed therein in order to deliver the desired formulation to the precisely desired location. In this way, a safe, effective, and reliable delivery system is realized.

In order to be certain that over-insertion of delivery nozzle/cannula 23 into a human is avoided, delivery nozzle/cannula 23 comprises any enlarged cap 28 formed at the proximal end of tube portion 25. By employing enlarged cap 28, a positive stop surface is provided, preventing accidental over-insertion of tube portion 25. In addition, this construction also prevents unwanted dislodgment of nozzle/cannula 23 and suction thereof into the cavity by the creation of a vacuum.

In addition, in the preferred embodiment, cap 28 comprises an interior threaded zone which is quickly and easily mounted to bottle 21 directly to threads formed thereon. In this way, delivery nozzle 23 is quickly mounted to bottle 21, after the desired composition 22 has been placed into bottle 21. As a result, the entire assembly is achieved both quickly and easily.

In accordance with the present invention, nozzle/cannula 23 may be molded with any desired length and diameter in order to satisfy the particular requirements of any desired application or use. In this regard, if required, the depth insertion guard can be eliminated.

Furthermore, in forming nozzle/cannula 23 of this invention, any bottle diameter, thread construction, or mounting system can be accommodated. In satisfying any such needs, nozzle/cannula 23 is molded with the desired configuration for achieving a particular construction or arrangement.

One common configuration found in the prior art for bottle 21 is the construction of bottle 21 with a flexible, thin-wall. By employing this type of bottle construction, a low-cost, extremely flexible bottle delivery system is realized. However, although bottles of this nature have been well received, due to their low cost and ease of use, control of the delivery pressure and the flow of the product through the delivery tube has been extremely difficult to properly regulate. As a result, either unsatisfactory flow control is produced or expensive valve constructions are required.

By employing the present invention, all of these difficulties and drawbacks have been completely eliminated, and an inexpensive product delivery system is realized, capable of providing complete control over the flow of formulation 22, without requiring expensive valve configurations. As best seen in FIG. 3, the present invention is constructed with tube portion 25 mounted in cap 28 with a small diameter aperture 29 communicating between the interior of bottle 21 and the open delivery channel formed in tube portion 25 through which composition 22 flows.

By controlling the diameter of aperture 29 to comprise a small diameter, consistent with the overall diameter of tube portion 25, complete control over the flow of product 22 through delivery tube 25 is automatically realized. By providing this construction, the requisite amount of back pressure is provided for enabling the user to effectively squeeze bottle 21 for delivery of product 22 through tube portion 25 in a consistent, efficient manner. Furthermore, by employing this construction, both ease of flow is attained as well as efficient delivery of product 22 through apertures 26.

In order to attain these highly desirable results, it has been found that tube portion 25 preferably comprises an outer diameter ranging between about 0.25 and 0.35 inches. Consistent with this overall outer diameter is the interior diameter of tube portion 25 which controls the dimension of aperture 29. In this regard, the preferred inside diameter of aperture 29 ranges between about 0.050 and 0.200 inches with a range of between about 0.080 and 0.156 being most desirable. By employing this construction, the desired flow characteristics and pressure control of squeeze bottle 21 are realized.

Furthermore, it has also been discovered that the delivery pressure is controlled by the size and number of apertures or slits 26 formed in delivery tube 25. Consequently, additional precise control over the performance of delivery system 20, as well as its delivery pressure, is realized by forming apertures/slits 26 in delivery tube 25 with a precise dimension as well as by controlling the number of apertures/slits 26 being employed. In this regard, nozzle/cannula 23 may be constructed with any desired thickness as well as with a smoothly curve, rounded end for comfort and ease, with a single aperture or slit 26 being formed in the rounded terminating end thereof.

In addition to employing the various product formulations 22 detailed herein for being dispensed by bottle 21 of delivery system 20 of FIG. 1, soft, flexible, readily compressible bottles 21 have typically been employed in the prior art for delivering douche products to individuals. It has been found that by employing delivery system 20 of FIG. 1 with a unique douche product formulated in accordance with this invention, a highly effective, therapeutic, and anti-viral treatment system is realized. In this regard, Tables X and XI provide preferred douche formulations which achieve both conventional cleaning and freshening, as well as providing therapeutic and anti-viral benefits.

TABLE X

| Ingredient | Preferred %/Wgt | Range %/Wgt |
|---|---|---|
| Deionized Water | 88.65 | q.s. to 100% |
| Citric Acid Powder, USP | 0.05 | 0.01-0.08 |
| Nonoxynol-9, USP or | 1.00 | 1.00-5.00 |
| Octoxylnol-9 | | |
| Glycerine, UPS | 10.00 | 8.0-12.00 |
| Povidone Iodine (#30/06) | 0.03 | 0.30-2.0 |
| Fragrance | As needed | As needed |

TABLE XI

| Ingredient | Range %/Wgt |
|---|---|
| Deionized Water/Local | 87.4 |
| Citric Acid Powder (USP (Spectrum Chemicals) | 0.1 |
| Hamposyl L-30 (Dow-Hempshire Chemicals) | 3.0 |
| Polyethylene Glycol #400 (Dow or equal) | 3.0 |
| Glycerine 96% USP (Dow or equal) | 5.0 |
| Povidone Iodine #30/06 (BASF Corp.) | 1.0 |
| Dibasic Sodium Phosphate, USP | 0.5 |
| (Spectrum Chemicals) | |
| | 100.0 |

In formulating the douche product defined in Table X, it is preferable to first add the deionized water to a standardized batch tank. Once the water extends over the mixing blades, moderate mixing should begin. Thereafter, each ingredient is added in the order provided in Table X, and the formulation is mixed for 30 minute in order to assure complete intermixing. Once fully mixed, this formulation is added to bottle 21 to provide the desired therapeutic and anti-viral douche system.

In preparing the douche product formulation defined in Table XI, it is preferable to first add the deionized water to a standardized batch tank which preferably incorporates a sanitized scale mounted therewith. Once the water extends over the mixing blades, moderate mixing should begin while heating of the water to 140° F.

Thereafter, each of the ingredients is added in the order provided in Table XI, except for the addition of povidone iodine. As each ingredient is added, heating and mixing continues, and, once all of the ingredients have been thoroughly intermixed with the temperature of the composition being held at about 140° F., heating is stopped and the povidine iodine is slowly sifted into the composition with mixing continuing. During the sifting step, the operator should have a face mask on for protection. Once the povidine iodine is completely in solution, the formulation is ready for cooling and subsequent addition to a desired container.

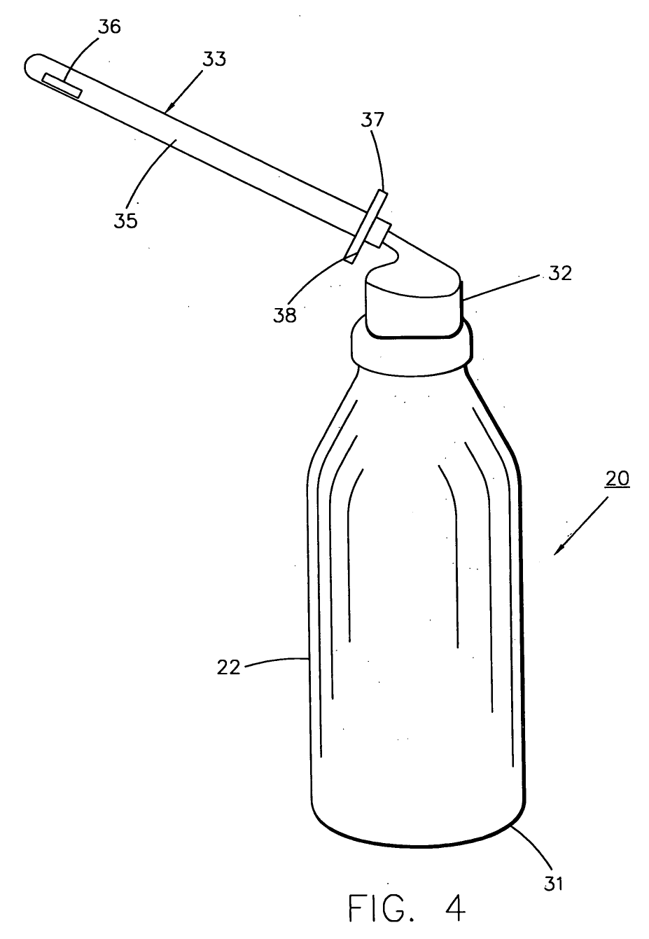

In FIG. 4, an alternate delivery system 20 made in accordance with the present invention is depicted. In this embodiment, bottle 31 is employed with the desired formulation 22 contained therein. In addition, in order to dispense product 22 from container 31, a finger operated, non-aerosol, pump valve 32 is employed by being affixed to the portal of bottle 31. In accordance with the present invention, virtually any desired, finger operated, pressure producing pump valve 32 may be employed. However, it has been found that high-pressure pump valves provide optimum results.

In accordance with the present invention, the construction of this embodiment of delivery system 20 is completed by inserting nozzle/cannula member 33 into the exit portal of valve 32. As fully detailed below, by employing this alternate embodiment, any desired formulation 22 is delivered precisely to the location where optimum efficacy is achieved.

In this embodiment, nozzle/cannula member 33 comprises an elongated tube portion 35 which is specifically configured for insertion into the desired human cavity, such as the vagina, the rectum, or deep wound. As with the embodiment detailed above, tube portion 35 comprises the precise overall length which assures complete insertion of tube portion 35 in the desired orifice or cavity, without injuring the surrounding soft tissue. As detailed above, this overall length typically comprises between about 2 inches and 3.5 inches.

As with the embodiment detailed above, the distal end of tube portion 35 comprises apertures 36 formed therein in order to enable the desired formulation to be uniformly dispensed precisely at the desired location. As a result, a safe, effective, and reliable delivery system is attained.

In this embodiment, in order to assure that over-insertion of nozzle/cannula member 33 into a human is avoided, nozzle/cannula member 33 incorporates an enlarged flange 37 formed adjacent the proximal end thereof for providing a positive stop surface. By incorporating flange 37, nozzle/cannula member 33 is incapable of being inserted beyond the precisely desired overall length.

The construction of nozzle/cannula member 33 is completed by forming proximal end 38 thereof in a manner which provides ease of engagement and securement in the portal of pump valve 32. By constructing proximal end 38 with an overall diameter that assures secure, frictional interengagement thereof in the portal of pump valve 32, an easily assembled, dependable and reliable delivery system 20 is realized. As is evident from this foregoing disclosure, the desired formulation 22 is easily dispensed at the precisely desired location in the human body by merely inserting nozzle/cannula member 33 into the desired cavity and then pressing pump valve 32 in order to dispense product formulation 22 to the precisely desired site.

As is evident from the foregoing detailed discussion, two alternate constructions for a highly effective and reliable product delivery system are attained by the present invention. By employing either of these alternate constructions, any desired formulation can be delivered by pressure to a precise interior location in the human body with ease and simplicity. Furthermore, both of these alternate embodiments enable any desired formulation to be delivered in a safe and effective manner to the precise sites or location where optimum beneficial results are attained. If desired, dip tubes can be employed with these embodiments for assisting in dispensing the desired formulation. In addition, both embodiments may be constructed with the bottles being re-fillable in order to achieve substantial economical benefits.

In addition to delivery of product formulations 22 under pressure, a further alternate method often employed is gravity feed. Although the nozzle/cannula constructions detailed above have been found to be equally effective in delivering any desired product using a gravity feed method, it has been found desirable to include a check valve or one-way valve formed in combination with the nozzle/cannula, in order to assure the delivery of the product to the desired location, without any chance of backwash or backflow being produced. As a result, a check valve or one-way valve construction is preferably incorporated into the nozzle/cannula of the present invention for gravity feed systems, particularly when using the present invention in the rectal area. In addition, if desired, a similar check valve or one-way valve can also be incorporated into the pressure delivery nozzle/cannula constructions detailed above in order to assure similar backflow or backwash problems are completely avoided.

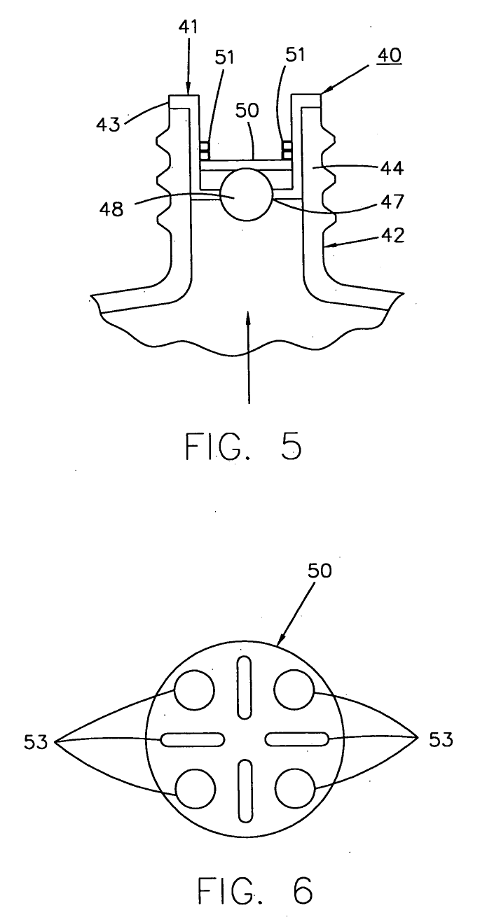

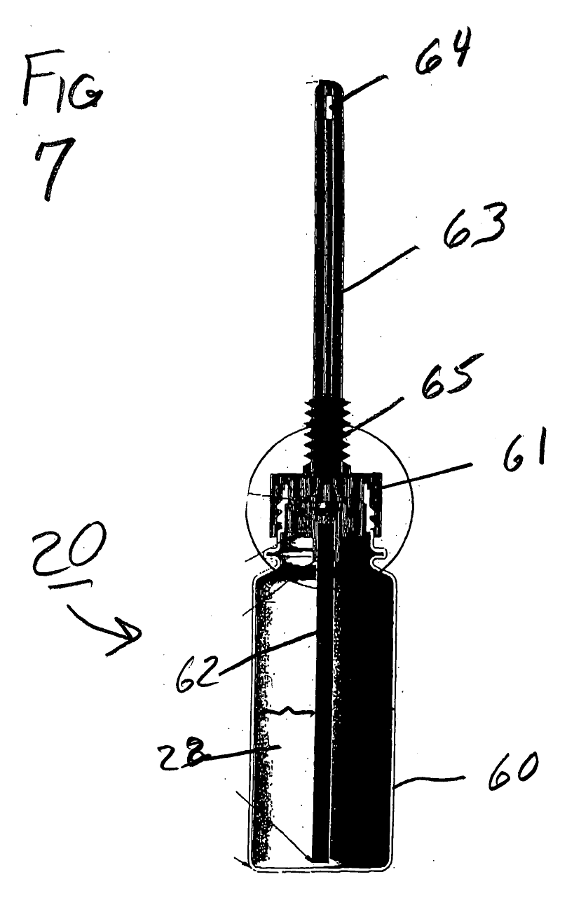

By referring to FIGS. 5 and 6, along with the following detailed discussion, a preferred construction for check valve 40 can best be understood. In this preferred embodiment, check valve 40 comprises cup member 41 which is inserted into neck or portal 44 of bottle 42. By forming cup member 41 with a radially extending flange 43 which peripherally surrounds the opened end thereof, cup member 41 is quickly and easily inserted into neck/portal 44 and securely retained in position. In addition, in order to complete the secure retention and placement of check valve 40, a suitable cap is threaded onto neck/portal 44 of bottle 41, securing cup member 41 between the cap and bottle 42.

In order to provide the desired controlled flow of the product retained in bottle 42, check valve 40 incorporates an aperture 47 formed in the base of cup member 41 and spherical ball 48 constructed for movable engagement with aperture 47. This construction is completed by providing movable disk 50 which cooperates with spherical ball 48 and cup member 41. In the preferred construction, disk 50 is capable of limited, controlled, axial movement within cup member 41, with said axial movement being limited between contact with spherical ball 48 and retaining ribs or detents 51 formed on the inside wall of cup member 41.

As clearly shown in FIG. 6, disk 50 comprises a plurality of apertures or through holes 53 formed therein in order to enable the composition retained in bottle 42 to easily pass through disk 50. However, before any product retained in bottle 42 is capable of flowing through check valve 40, spherical ball 48 must be dislodged from aperture 47.

As is evident from the foregoing detailed discussion, whenever pressure is applied to bottle 42, or bottle 42 is inverted to cause gravity flow of the composition retained therein, spherical ball 48 is dislodged from aperture 47, enabling the composition retained in bottle 42 to flow through aperture 47, around ball 48, and through apertures or portals 53 formed in disk 50. The required movement of disk 50 which enables spherical ball 48 to be dislodged from aperture 47 is provided by the limited axial movement of disk 50 within cup member 41 between ball 48 and ribs/detents 51.

In addition, whenever activation pressure is removed from bottle 41, or reverse flow is experienced from the delivery side of check valve 40, disk 50 returns to its original position, forcing spherical ball 48 into aperture 47. As a result, flow into bottle 42 from the delivery source is stopped and contamination of the composition in bottle 41 is prevented. In this way, all of the attributes sought to be achieved in the delivery of a composition in accordance with the present invention are realized, either pre or post coitus.

In FIG. 7, a further alternate embodiment of delivery system 20 of the present invention is depicted. In this embodiment, delivery system 20 comprises flexible bottle 60 which incorporates cap 61 threadedly mounted to the open portal of bottle 60, thereby securely retaining the desired product formulation 22 within bottle 60. In addition, dip tube 62 is mounted to cap 61 extending therefrom into product formulation 22, in order to easily draw product formulation 22 in cap 61.

In addition, as depicted, nozzle/cannula 63 is affixed to cap 21 extending therefrom in order to deliver the desired formulation directly to the precise location where formulation 22 is desired. In this regard, nozzle/cannula 63 is constructed in the substantially identical manner as detailed above, thereby providing the efficient and to safe delivery of product formulation 22 to the precisely desired location. Furthermore, as depicted, nozzle/cannula 63 incorporates aperture 64 formed at the distal end thereof for assuring the delivery of product 22 at the precisely desired site. Although aperture 64 is depicted as a slot, any desired configuration can be employed, including a single or multiple, generally circular shaped apertures or passageways.

In FIG. 7, nozzle/cannula 63 is shown mounted to cap 21 in combination with bellows configuration 65. This configuration is shown for exemplary purposes, and is not required. However, if desired, bellows 65 can be employed in order to add a greater flexibility and arcuate movement to nozzle/cannula 63 relative to cap 61.

One particular feature provided by this embodiment of delivery system 20, which has not been depicted or exemplified in the earlier embodiments, is the incorporation in cap 61 of independent airflow passageways which communicate between the ambient surroundings and the interior of bottle 60. In this way, assurances provided that unwanted backflow through nozzle/cannula 63 is avoided.

In operation, a user compresses or squeezes bottle 60 in order to force product formulation 22 through dip tube 62 and cap 61 for creating the desired foam and forcing the foam through nozzle/cannula 63. Once the foamed product 22 has passed through the entire length of nozzle/cannula 63, the foamed product 22 is dispensed through aperture 64. Thereafter, the user removes the compressive forces from bottle 60 for enabling bottle 60 to return to its original configuration.

In order to assure that a backflow or suction force is not created for drawing material back through nozzle/cannula 63, cap 61 incorporates airflow passageways formed therein which enables ambient air to pass through cap 61 into the interior of bottle 60, whereby bottle 60 is able to return to its original configuration. By employing this construction, contamination of the interior of nozzle/cannula 63 is prevented and continuous operation and fast and convenient dispensing of the desired product at the precisely desired site is achieved.

A further area in which it has been found that the present invention is highly effective in providing a therapeutic treatment system, which presently does not exist, is in treating or reducing the transmission of viral diseases, such as AIDS, herpes, and chlamydia. By employing the present invention with a suitable therapeutic agent incorporated therein, a prophylactic treatment system is realized for helping to prevent the transmission of these viral diseases.

In order to attain an effective anti-viral treatment system employing the liquid foam producing compositions of the present invention, it has been found that the therapeutic agent incorporated into the composition is preferably selected from the group consisting of spirulina, calcium-spirulan, nonoxynol-9, octoxynol-9, hot water solution of spirulan, povidone iodine or iodine salt, silver nanocrystals and colloidal silver. These compounds, along with other compounds being developed having equal efficacy, can be employed in the present invention in order to provide the desired prophylactic results.

Recently, it has been found that these compounds are capable of inhibiting viral replication, while strengthening both the cellular and hormonal arms of the immune system, causing regression and inhibition of various diseases. As a result, these compounds, and their equivalents, provide effective therapeutic agents for being incorporated in the liquid foam producing compositions of the present invention in order to provide an easily used, highly effective medicinal delivery system for helping to reduce transmission of viral diseases such as AIDS, herpes, chlamydia and gonorrhea. In addition, by employing these formulations using the delivery system detailed above, a highly effective and easily employed product is realized.

The invention accordingly comprises a composition of matter possessing the characteristics, properties, and the relation of constituents which will be exemplified in the compositions hereinafter described, and the scope of the invention will be indicated in the claims.

### BEST MODE FOR CARRYING OUT THE INVENTION

By referring to the following detailed disclosure, various preferred constructions and formulations of the liquid foam producing product of the present invention, and the production of such compositions can best be understood. Although the following disclosure specifically details alternate formulations for the liquid foam producing compositions, as well as preferred methods for creating the compositions, alternate formulations and methods can be employed without departing from the scope of this invention. Consequently, it is to be understood that the following specific formulations and methods are provided for exemplary purposes and any alternate formulations and production methods coming within the scope of the present invention are intended to be encompassed therein.

In accordance with the present invention, multi-purpose, antiseptic, anti-bacterial, and/or anti-viral liquid foam producing formulations capable of providing a thick, rich, moisture laden foam mousse are attained. By referring to Tables XII-XVI, several alternate preferred, specific formulations for this multi-purpose, antiseptic, anti-bacterial, and/or anti-viral improved liquid foam producing product are provided. Although these formulations may be varied or altered, Tables XI-XVI provide several preferred, specific formulation for a multi-purpose, foam producing composition made in accordance with the present invention.

TABLE XII
Triclosan Based Foam Producing Composition

| Ingredient | % by Weight |
|---|---|
| Polysorbate 20 | 10 |
| Ammonium Lauryl Sulfate | 30 |
| Cocoamide DEA | 5 |
| Triclosan | 0.2 |
| Water | 54.8 |

TABLE XIII
Colloidal Silver Based Foam Producing Composition

| Ingredient | % by Weight |
|---|---|
| Colloidal Silver Solution | 75-99.8 |
| (10 ppm-32 ppm in water) | |
| Surfactants | 0.1-25 |
| Additives | 0.5-1 |

TABLE XIV
Nanocrystalline Based Foam Producing Composition

| Ingredient | % by Weight |
|---|---|
| Silver Nanocrystalline Powder | 0.1 |
| Propylene Glycol, USP | 5.0 |
| Denatured Ethanol | 20 |
| Sodium Lauroyl Sarcosinate | 1.0 |
| Water | q.s. to 100% |

TABLE XV
Nanocrystalline Based Foam Producing Composition

| Ingredient | % by Weight |
|---|---|
| Silver Nanocrystalline Powder | 0.1 |
| Sodium Lauroyl Sarcosinate | 4.0 |
| Deionized Water | q.s. to 100% |

TABLE XVI
Nanocrystalline Based Foam Producing Composition

| Ingredient | % by Weight |
|---|---|
| Silver Nanocrystalline Powder | 0.1 |
| Propylene Glycol, USP | 5.0 |
| Denatured Ethanol | 30 |
| Deionized Water | q.s. to 100% |

In order to attain a thoroughly intermixed, substantially homogeneous, multi-purpose improved liquid foam producing composition employing the ingredients defined in Table XII, it has been found that a preferred mixing process be employed. In this regard, polysorbate 20 and triclosan are added to a first vessel, heated to about 65° C. and continuously mixed together until fully dissolved. In addition, ammonium lauryl sulfate and cocoamide DEA are intermixed in a second vessel and heated to about 65° C. These components are also intermixed until fully dissolved.

In the next process step, polysorbate 20 triclosan mixture is added to the ammonium lauryl sulfate and cocoamide DEA combination and the resulting composition is thoroughly mixed. Then, the water is added to the component mixture and the entire mixture is heated to a temperature of about 50° C. Once heated, thorough intermixing of all components is provided, and when completed, the resulting composition is allowed to cool to room temperature.

It has been found that the resulting composition produced by employing the foregoing process typically has a pH of about 8.4. In order to lower the pH to between about 6.5 and 8.0, a desired pH adjusting agent is employed. Typically, citric acid or mild H.L. is effectively used for this purpose. By employing this process, or an equivalent process, a highly effective, multi-purpose, antiseptic, antibacterial, and/or antiviral liquid foaming soap composition is realized.

Whenever one of the preferred multi-purpose formulations detailed in Tables XII-XVI are complete, the resulting composition is placed in a container which is then interconnected with a movable, finger operated foam dispensing cap/valve or a foam producing cap member which relies upon squeezing of the container for forcing the composition therethrough. Preferably, if this latter construction is employed, the foam producing cap member incorporates an elongated, soft, pliable, nozzle member mounted thereto for enabling the delivery of the foam product directly to any desired site or location, such as an internal site located within a body cavity.

It has been found that by employing the present invention, a highly desirable, multi-purpose, antiseptic, anti-bacterial, and/or anti-viral liquid based foam producing product is attained, which can be used for virtually any desired medical or medicinal purpose and/or everyday cleansing operations. In addition, by employing an effective amount of silver as defined in Tables XIII-XVI, a highly desirable, easily employed, multi-purpose foam producing product is realized which is capable of delivering an anti-bacteria, anti-virus, anti-fungal agent to any specific location for treating a specific medical need. In addition, this foam product may also be used as a mouth wash, mouth rinse, on face masks, air filter sheets, and/or on sheets used as wipes or breathing masks.

As detailed above, one example of such medical purposes is the reduction in the spreading or transmission of diseases such as AIDS, herpes, and chlamydia by employing an anti-viral agent such as nonoxynol-9, spirulines, calcium spiruline and hot water solutions or spirulan. Furthermore, a liquid-based foam producing formulations particularly suitable for assisting in the treatment of burn victims is realized by employing povidone-iodine or silver nitrate solutions as the therapeutic agent. Finally, colloidal silver, colloidal silver solutions, or silver nanocrystals can be employed to provide a foam product having unique anti-viral, anti-bacterial and anti-fungal properties which is employable for controlling a wide variety of diseases and medical problems.

Although specific alternate formulations for compositions employing alternate therapeutic agents are not specifically provided herein, such alternate formulations are clearly within the teaching of the present invention by employing the formulations and production steps detailed above. As a result, the incorporation of virtually any therapeutic agent into the basic formulations of the present invention is clearly within the scope of this invention and is intended to be encompassed by the claims of this invention.

In Tables XIV-XVI, alternate formulations employing silver nanocrystalline powder to achieve a foam producing composition are fully detailed. Each of these formulations were independently prepared and independently tested. Based upon the test results of each experiment, it was found that the foam composition produced by these formulations were capable of completely eradicating particular bacteria, fungi, and viruses to which the foam was applied during the testing procedures.

In Tables XIV-XVI, the formulations detailed therein are formed for being dispensed by a finger operated pump dispenser as a thick, rich foam mousse. The composition defined in Table XVI employs a propellent for producing foam mousse dispensed as an aerosol. In all of these applications, the foam composition dispensed therefrom proved to be highly effective in eradicating the desired microbes.

In order to define a further alternate preferred formulation of the all natural, foam producing, improved product of the present invention, Table XVII is provided. In Table XVII, the preferred additives and enhancing agents are fully disclosed. In Table XVII, each of the ingredients are defined in a preferred quantity range, with the amount provided as a percent by weight, based upon the weight of the entire composition.

TABLE XVII

| | Quantity |
|---|---|
| Ingredients | % by Weight |
| Stearic Acid | 2-20 |
| Potassium Cocotte Acids | 10-40 |
| Glycerin USP | 0.5-5 |
| Water | 50-90 |
| Citronellol | 0.5-1.0 |
| pH Adjusting Agent | 2-10 |
| Therapeutic Agent | Effective Amount |

In carrying out the present invention, an all-natural, liquid foam producing formulation is produced consistent with the formulations defined above, with the liquid foam producing formulation being placed in a conventional liquid product holding container. Since pressurization of the composition is not required, the composition is retained in the container at conventional, atmospheric conditions, and any suitable container normally employed for such a liquid product can be used.

Once a suitable quantity of the all natural, liquid foam producing formulation of the present invention is added to the desired container, the container is closed by mounting a suitable foam producing, dispensing valve or cap to the open portal of the container. In accordance with the present invention, the foam producing dispensing valve or cap mounted to the container within which the improved liquid foam producing composition is retained may comprise a wide variety of alternate constructions. However, regardless of the precise configuration employed, a suitable foam producing dispensing valve or cap should be capable of drawing the improved liquid foam producing composition into the cap or valve and mixing with air, dispensing the desired foam mousse. Typically, this process includes compressing the improved composition in the valve while infusing air into the formulation prior to dispensing the mousse. However, any alternate process can be employed with equal efficacy.

As discussed above, in an alternate embodiment of the present invention, the desired foam shaving mousse is obtained by employing colloidal silver in combination with an all natural liquid soap formed from vegetable oil base which has been split using steam. In this embodiment, the vegetable oil base employed is preferably selected from the group consisting of palm kernel oil and coconut oil. For the neutralization of the generated fatty acid, potassium hydroxide has been used. In order to obtain the desired liquid soap formulation, the partially neutralized fatty acids are intermixed with water, heated and agitated. In addition, the pH is adjusted until a pH of between about 9.2 and 10 is achieved. If desired, pH adjusting agents, enchanters, fragrances, and preservatives may be added to the formulation to obtain the final liquid foaming soap product of the present invention. In the preferred embodiment, these additives include aloe vera, rosemary extracts, citric acid, a blend of natural essential oils, and lanolin.

In the preferred formulation, based upon the weight of the entire composition, the vegetable oil based ingredients ranges between about 30% and 60% of the weight and water makes up about 50% to 90% by weight. These quantities are reduced by the quantity employed for any additive which is desired.

As discussed above, the pH of this embodiment of the liquid foam soap composition is preferably maintained between about 8.8 and 10. More preferably, it has been found that a pH of between about 9.2 and 9.4 is most desirable. It has been found that at a pH level below 9.2, the ingredients may separate and/or may require the inclusion of a preservative.

Furthermore, as discussed above, the foam generating ingredients are added to water, heated and agitated in order to obtain the desired fully intermixed composition. In this regard, it has been found that raising the temperature of the composition to between about 55° C. and 75° C. provides the desired intermixing, in a manner which is most expeditious.

As discussed above, once a desired composition has been obtained, a suitable quantity of the composition is added to a liquid holding container, with the container being closed by a foam dispensing valve or cap. In this way, the desired, all natural, foam mousse is produced whenever a consumer activates the valve or cap, generating the desired thick, rich, moisture laden foam for application to any skin surface or external site.

It will thus be seen that the object set forth above, among those made apparent from the preceding description, are efficiently attained and, since certain changes may be made in the above product without departing from the scope of the invention, it is intended that all matter contained in the above description shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Particularly, it is to be understood that in the claims, ingredients or compounds recited in the singular are intended to include compatible mixtures of such ingredients wherever the sense permits.

Having described our invention, what I claim as new and desire to secure by letters patent is:

1. A medicinal delivery system for enabling the application of an antibacterial/antiviral foam producing composition to a topical or internal site of an individual, said delivery system comprising:
A. an antibacterial/antiviral/antimicrobial foam producing composition comprising
a. between about 0.1% and 60% by weight based upon the weight composition of at least one surfactant selected from the group consisting of polysorbate 20, cocamide DEA, polysorbate 60, polysorbate 80, ammonium or alkaline salts of sulfated aliphatic alcohols, ammonium or alkaline salts of sulfated aliphatic ethoxylated alcohols, cocoamido derivatives, ethoxylate aliphatic phenolics, sarcosinates, sodium lauryl sulfoacetate, sodium lauryol sarcosinate, and vegetable oil based soaps;
b. an effective amount of at least one therapeutic agent selected from the group consisting of silver nitrate solutions, silver nanocrystals, colloidal silver, colloidal silver solutions, and equivalents thereof, and
c. water forming the balance; and
B. a container for dispensing the therapeutic agent as an integral component of the foam mousse for direct application of the foam mousse to the skin and/or an internal site of an individual, said container comprising one selected from the group consisting of finger actuated foam producing valve bearing containers and squeeze bottle foam producing containers.

2. The medicinal delivery system defined in claim 1, wherein the therapeutic agent is further defined as comprising a combined solution of colloidal silver in water, with the colloidal silver comprising between about 10 and 32 parts per million.

3. The medicinal delivery system defined in claim 2, wherein the therapeutic agent comprises between about 40% and 99.8% by weight based upon the weight of the entire compositions.

4. The medicinal delivery system defined in claim 3, wherein the surfactant is further defined as comprising between about 0.1% and 30% by weight based upon the weight of the entire composition of at least one selected from the group consisting of sodium lauryl sulfoacetate, sodium lauroyl sarcosinate, and vegetable oil based soap.

5. The medicinal delivery system defined in claim 4, wherein the combined solution of colloidal silver in water is further defined as comprising between about 99% and 99.8% by weight based upon the weight of the entire composition.

6. The medicinal delivery system defined in claim 5, wherein the surfactant is further defined as comprising between about 0.2% and 1.0% by weight based upon the weight of the entire composition of at least one selected from the group consisting of sodium lauryl surfaocetate and sodium lauroyl sarcosinate.

7. The medicinal delivery system defined in claim 1, wherein said therapeutic agent is further defined as comprising between about 0.05% and 1% by weight based upon the weight of the entire composition of silver nanocrystal powder.

8. The medicinal delivery system defined in claim 7, wherein the surfactant is further defined as comprising between about 0.5% and 5% by weight based upon the weight of the entire composition of one selected from the group consisting of sodium lauryl sulfoacetate and sodium lauroyl sarcosinate.

9. The medicinal delivery system defined in claim 8, wherein the foam producing composition further comprises between about 20% and 35% by weight based upon the weight of the entire composition of at least one additive.

10. The medicinal delivery system defined in claim 9, wherein said additive is further defined as comprising one selected from the group consisting of propylene glycol and denatured ethanol.

11. The medicinal delivery system defined in claim 1, wherein said system further comprises:
C. a delivery nozzle or cannula mounted to the container and comprising:
a. an elongated tube portion having an overall length constructed for ease of insertion into an orifice communicating with a cavity of the human body, said overall length being constructed to assure the complete insertion of the tube portion into the cavity without injuring any surrounding tissue; and
b. at least one aperture formed along the distal end of said elongated tube portion for enabling the medicinal foam composition to be dispensed from the container through the tube portion directly to the desired site.

12. The medicinal delivery system defined in claim 11, wherein said delivery nozzle or cannula further comprises an enlarged surface formed adjacent the proximal end thereof, for providing a positive stop for said tube member in order to prevent over-insertion of said tube member into the cavity.

13. The delivery system and defined in claim 11, wherein said elongated tube portion is further defined as being formed from flexible material in order to assure ease of use and insertion thereof into the human body for being positioned in the precisely desired location.

14. The delivery system defined in claim 11, wherein said container further comprises closure means comprising one selected from the group consisting of caps and finger-operated dispensing valves, and the delivery nozzle/cannula is further defined as being securely affixed to said closure means in a manner which prevents dislodgement thereof from said closure means.

15. The delivery system defined in claim 14, wherein the elongated tube portion of the delivery nozzle/cannula is further defined as comprising a tapered outer surface with a smaller diameter section being formed at the distal end thereof for further assisting in providing ease of insertion and positioning of the tube member in the desired orifice.

16. The delivery system defined in claim 14, wherein the elongated tube portion of the delivery nozzle/cannula is further defined as comprising a substantially uniform diameter throughout the entire length thereof, with the distal end of said tube portion being smoothly rounded to assure ease of insertion into the desired orifice without injuring any surrounding tissue.

17. The delivery system defined in claim 11, wherein said container is further defined as comprising a thin walled, flexible construction and said closure means comprises a cap member affixed to the portal of the container for enabling the foam producing medicinal composition in said container to be dispensed directly to the desired internal site in the human body by inserting the elongated tube portion into an orifice of the human body, advancing the tube portion into the cavity associated therewith and enabling the foam producing medicinal composition to be dispensed directly to the desired site as a foam mousse in response to pressure 15 being applied to the thin walled, flexible container.

18. The delivery system defined in claim 17, wherein the elongated tube portion comprises an elongated delivery channel formed by the internal diameter thereof which is constructed for controlling the delivery pressure produced by squeezing the flexible container.

19. An all natural foam mousse producing medicinal composition constructed for use on an individual's skin surface and/or internally, said composition comprising
A. between about 100% and 50% by weight based upon the weight of the entire composition of a vegetable oil based soap;
B. between about 50% and 90% by weight based upon the weight of the entire composition of water;
C. between about 0.3% and 2% by weight based upon the weight of the entire composition of lanolin; and
D. an effective amount of at least one therapeutical agent selected from the group consisting of silver nanocrystals, silver nitrate solutions, colloidal silver, colloidal silver solutions, and equivalents thereof.

20. The all natural foam mousse producing composition defined in claim 19, wherein the vegetable oil based soap is further defined as comprising one selected from the group consisting of palm kernel oil and coconut oil.

21. The all natural foam mousse producing composition defined in claim 19, wherein the therapeutic agent is further defined as comprising a combined solution of colloidal silver in water, with the colloidal silver ranging between about 10 and 32 parts per million.

22. A method for providing the delivery of a medicinal, anti-bacterial/antiviral foam mousse composition to a desired site of an individual comprising the steps of:
A. dispensing a medicinal foam mousse from a container housing a medicinal foam producing composition, said composition comprising
a. between about 0.1% and 60% by weight based upon the weight composition of at least one surfactant selected from the group consisting of polysorbate 20, cocamide DEA, polysorbate 60, polysorbate 80, ammonium or alkaline salts of sulfated aliphatic alcohols, ammonium or alkaline salts of sulfated aliphatic ethoxylated alcohols, cocoamido derivatives and ethoxylate aliphatic phenolics, sarcosinates, sodium lauryl sulfoacetate, sodium lauroyl sarcosinate, and vegetable oil based soaps;
b. an effective amount of at least one therapeutic agent selected from the group consisting of silver nitrate solutions, silver nanocrystals, colloidal silver, colloidal silver solutions, and equivalents thereof, and
c. water forming the balance; and
B. applying the medicinal foam mousse onto a desired surface; whereby the desired surface is imparted with an antimicrobial treatment for reducing and/or killing bacteria, viruses, funguses, and the like.

23. The method defined in claim 22, wherein said surface is further defined as comprising one selected from the group consisting of skin, human tissue, human cells, fabric, filters, carrier sheets, non-woven material, woven material, plastic material, medical devices, stints, bandages, sutures, and the like.

24. The method defined in claim 23, wherein the application step is achieved by spraying the foam mousse onto a desired surface.

25. The method defined in claim 23, wherein the therapeutic agent comprises colloidal silver solutions and the colloidal silver solution is applied to the desired surface by employing one process selected from the group consisting of spraying and dipping and then drying prior to use.

26. An antibacterial/antiviral/antimicrobial foam producing composition comprising:
A. between about 0.1% and 60% by weight based upon the weight composition of at least one surfactant selected from the group consisting of polysorbate 20, cocamide DEA, polysorbate 60, polysorbate 80, ammonium or alkaline salts of sulfated aliphatic alcohols, ammonium or alkaline salts of sulfated aliphatic ethoxylated alcohols, cocoamido derivatives, ethoxylate aliphatic phenolics, sarcosinates, sodium lauryl sulfoacetate, sodium lauryol sarcosinate, and vegetable oil based soaps;
B. an effective amount of at least one therapeutic agent selected from the group consisting of silver nitrate solutions, silver nanocrystals, colloidal silver, colloidal silver solutions, and equivalents thereof, and
C. water forming the balance.

27. The medicinal delivery system defined in claim 26, wherein the therapeutic agent is further defined as comprising a combined solution of colloidal silver in water, with the colloidal silver comprising between about 10 and 32 parts per million and between about 40% and 99.8% by weight based upon the weight of the entire compositions.

28. The medicinal delivery system defined in claim 26, wherein the surfactant is further defined as comprising between about 0.1% and 30% by weight based upon the weight of the entire composition of at least one selected from the group consisting of sodium lauryl sulfoacetate, sodium lauroyl sarcosinate, and vegetable oil based soap.

29. The medicinal delivery system defined in claim 26, wherein said therapeutic agent is further defined as comprising between about 0.05% and 1% by weight based upon the weight of the entire composition of silver nanocrystal powder. 30. The medicinal delivery system defined in claim 29, wherein the surfactant is further defined as comprising between about 0.5% and 5% by weight based upon the weight of the entire composition of one selected from the group consisting of sodium lauryl sulfoacetate and sodium lauroyl sarcosinate.
